(19) United States
(12) Patent Application Publication
(10) Pub. No.: US 2015/0265585 A1
(43) Pub. Date: Sep. 24, 2015

(54) Method of Treating Skin Disorders

(71) Applicant: RCP Development, Inc., Sarasota, FL (US)

(72) Inventor: Jonnie R. Williams, Sarasota, FL (US)

(73) Assignee: RCP DEVELOPMENT, INC., Sarasota, FL (US)

(21) Appl. No.: 14/729,605

(22) Filed: Jun. 3, 2015

Related U.S. Application Data
(62) Division of application No. 13/803,028, filed on Mar. 14, 2013.
Application No. 13/803,028 is a continuation-in-part of application No. 13/235,860, filed on Sep. 19, 2011.
(60) Provisional application No. 61/383,811, filed on Sep. 17, 2010.
(60) Provisional application No. 61/384,447, filed on Sep. 20, 2010.
(60) Provisional application No. 61/439,473, filed on Feb. 4, 2011.
(60) Provisional application No. 61/480,271, filed on Apr. 28, 2011.
(60) Provisional application No. 61/480,258, filed on Apr. 28, 2011.

(51) Int. Cl. A61K 31/444; A61K 9/00

(52) U.S. Cl. CPC A61K 31/444; A61K 9/0014

(57) ABSTRACT

A skin care product includes a compound of Formula I, IA, or IB (e.g., anatabine), or a salt thereof, and a suitable cosmetic or pharmaceutical vehicle. In some aspects, the skin care product is a paste, cream, lotion, gel, moisturizer, cleanser, or sunscreen. In some embodiments, the skin care product is applied topically to reduce inflammation, redness, and/or irritation, and/or to improve the appearance of the skin. In other embodiments, the skin care product is applied topically to treat an autoimmune and/or dermatological condition such as acne, psoriasis, and/or rosacea.

### CROSS REFERENCE TO RELATED APPLICATIONS

This application is a division of application Ser. No. 13/803,028, filed Mar. 14, 2013, which is a continuation-in-part of application Ser. No. 13/235,860, filed Sep. 19, 2011, which claims the benefit of App. No. 61/383,811 filed Sep. 17, 2010; App. No. 61/384,447 filed Sep. 20, 2010; App. No. 61/439,473 filed Feb. 4, 2011; App. No. 61/480,271 filed Apr. 28, 2011; and App. No. 61/480,258 filed Apr. 28, 2011. The Ser. No. 13/803,028 application also is a continuation-in-part of application Ser. No. 13/494,237, filed Jun. 12, 2012, which claims the benefit of App. No. 61/528,380 filed Aug. 29, 2011. Each of the applications identified above is hereby incorporated by reference in its entirety.

### SUMMARY

A skin care product comprises a compound of Formula I, IA, or IB (e.g., anatabine, S-(−)-anatabine, or R-(+)-anatabine) or a salt thereof, and a suitable cosmetic or pharmaceutical vehicle. In some aspects, the skin care product is a paste, cream, lotion, gel, moisturizer, cleanser, or sunscreen. In some aspects, the skin care product may be applied topically to reduce inflammation, redness, or irritation, and/or to improve the appearance of the skin. For example, a skin care product may be topically administered to reduce the appearance of redness and/or to reduce the appearance of dark circles on the skin. In other aspects, the skin care product may be applied topically to treat an autoimmune and/or dermatological condition such as acne, psoriasis, and/or rosacea.

### DETAILED DESCRIPTION

Skin care products may contain an isolated form of a compound of Formula I or IA (e.g., anatabine, S-(−)-anatabine, or R-(+)-anatabine) or a salt thereof. As described in the present inventor's Published Application U.S. 2012/0245202 A1, such compounds are useful for, among other things, maintaining inflammation at levels that promote well-being. As used herein, the term “skin care product” refers to solid, semisolid, or liquid formulations suitable for topical application, particularly to the skin, and in some cases may be categorized as pharmaceutical or cosmetic type products.

Compounds of Formula I

In some embodiments, a composition comprises an isolated form of a compound of Formula I, which can be provided as a pharmaceutically acceptable or food-grade salt:

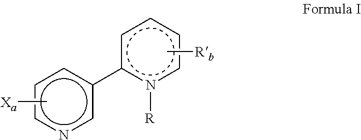

wherein:
R represents hydrogen or C₁-C₅ alkyl;
R′ represents hydrogen or C₁-C₇ alkyl; and
X represents halogen or C₁-C₇ alkyl.

In some embodiments,
R represents hydrogen or C₁-C₃ alkyl;
R′ represents hydrogen or C₁-C₄ alkyl; and
X represents halogen or C₁-C₃ alkyl.

The dotted line within the piperidine ring represents a carbon/carbon or carbon/nitrogen double bond within that ring, or two conjugated double bonds within that ring. One of the two conjugated double bonds can be a carbon/nitrogen double bond, or both of the conjugated double bonds can be carbon/carbon double bonds. When a carbon/nitrogen double bond is present, R is absent; and either (i) “a” is an integer ranging from 1-4, usually 1-2, and “b” is an integer ranging from 0-8, usually 0-4; or (ii) “a” is an integer ranging from 0-4, usually 0-2, and “b” is an integer ranging from 1-8, usually 1-4. When a carbon/nitrogen double bond is not present, R is present; “a” is an integer ranging from 0-4, usually 1-2; and “b” is an integer ranging from 0-8, usually 0-4 or 1-2. The term “alkyl,” as used herein, encompasses both straight chain and branched alkyl. The term “halogen” encompasses fluorine (F), chlorine (Cl), bromine (Br), and iodine (I).

Table 1 below illustrates non-limiting examples of compounds within Formula I:

TABLE 1

| R | R′ (position) | X (position) | a | b |
|---|---|---|---|---|
| H | CH₃ (3) | — | 0 | 1 |
| CH₃ | — | CH₃ (5) | 1 | 0 |
| H | — | CH₃CH₂ (4) | 1 | 0 |
| CH₃CH₂ | CH₃ (4) | — | 0 | 1 |
| H | CH₃ (2) | — | 0 | 2 |
| | CH₃CH₂ (5) | | | |
| H | CH₃ (3) | CH₃ (5) | 1 | 1 |
| CH₃ | — | CH₃ (2) | 2 | 0 |
| | | CH₃ (5) | | |

Compounds of Formula I may be present in the form of racemic mixtures or, in some cases, as isolated enantiomers as illustrated below in Formulas IA and IB.

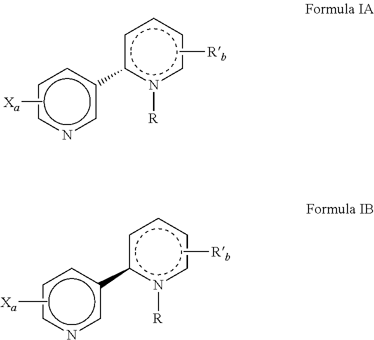

An example of a compound of Formula I is anatabine. An example of a compound of Formula IA is S-(−)-anatabine, and an example of compound of Formula IB is R-(+)-anatabine.

The chemical structure of anatabine (1,2,3,6-tetrahydro-[2,3′]bipyridinyl) is illustrated below, in which * designates an asymmetric carbon.

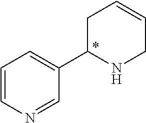

Anatabine exists in tobacco and certain foods and plants, including green tomatoes, green potatoes, ripe red peppers, tomatillos, sundried tomatoes, datura, mandrake, belladonna, capsicum, eggplant, and petunia, as a mixture of R-(+)-anatabine and S-(−)-anatabine, whose structures are illustrated below.

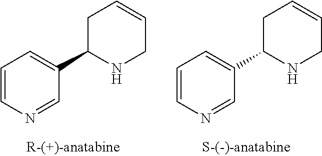

Anatabine, R-(+)-anatabine, S-(−)-anatabine, and other compounds of Formula I, IA, and IB can be prepared synthetically. Such synthetic preparation techniques produce isolated forms of the compounds. Methods for selectively preparing the anatabine enantiomers are described, for example, in “A General Procedure for the Enantioselective Synthesis of the Minor Tobacco Alkaloids Nornicotine, Anabasine, and Anatabine,” The AAPS Journal 2005; 7(3) Article 75.

Anatabine may be prepared via a benzophenoneimine pathway, as described in commonly owned U.S. Pat. No. 8,207,346, the disclosure of which is incorporated herein by reference in its entirety.

In some embodiments, a compound of Formula I, IA, or IB (e.g., anatabine, S-(−)-anatabine, or R-(+)-anatabine) may be adsorbed on a cation exchange resin such as polymethacrilic acid (Amberlite IRP64 or Purolite C115HMR), as described in U.S. Pat. No. 3,901,248, the disclosure of which is hereby incorporated by reference in its entirety. Such cation exchange resins have been used commercially, for example, in nicotine replacement therapy, e.g., nicotine polacrilex.

In some embodiments, a compound of Formula I, IA, or IB (e.g., anatabine, S-(−)-anatabine, or R-(+)-anatabine) is provided in the form of a salt. “Salt,” as used herein, includes pharmaceutically acceptable and food-grade salts. In general, salts may provide improved chemical purity, stability, solubility, and/or bioavailability relative to anatabine in its native form. Non-limiting examples of possible anatabine salts are described in P. H. Stahl et al., Handbook of Pharmaceutical Salts: Properties, Selection and Use, Weinheim/Zürich:Wiley-VCH/VHCA, 2002, including salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid.

As an alternative to preparing anatabine synthetically, anatabine can be obtained by extraction from tobacco or other plants, such as members of the Solanaceae family, such as datura, mandrake, belladonna, capsicum, potato, nicotiana, eggplant, and petunia. For example, a tobacco extract may be prepared from cured tobacco stems, lamina, or both. In the extraction process, cured tobacco material is extracted with a solvent, typically water, ethanol, steam, or carbon dioxide. The resulting solution contains the soluble components of the tobacco, including anatabine. Anatabine may be purified from the other components of the tobacco using suitable techniques such as liquid chromatography.

As part of the purification process, tobacco material may be substantially denicotinized to remove a majority of other alkaloids such as nicotine, nornicotine, and anabasine. Denicotinizing is usually carried out prior to extraction of anatabine. Methods that may be used for denicotinizing tobacco materials are described, for example, in U.S. Pat. No. 5,119,835, the disclosure of which is hereby incorporated by reference. In general, tobacco alkaloids may be extracted from tobacco material with carbon dioxide under supercritical conditions. The tobacco alkaloids may then be separated from the carbon dioxide by dissolving an organic acid or a salt thereof, such as potassium monocitrate, in the carbon dioxide.

In some embodiments, an isolated form of anatabine is used. An “isolated form of anatabine,” as used herein, refers to anatabine that either has been prepared synthetically or has been substantially separated from plant materials in which it occurs naturally. The isolated form of anatabine should have a very high purity (including enantiomeric purity in the case where an enantiomer is used). In the case of synthetic anatabine, for example, purity refers to the ratio of the weight of anatabine to the weight of the end reaction product. In the case of isolating anatabine from plant material, for example, purity refers to the ratio of the weight of anatabine to the total weight of the anatabine-containing extract. Usually, the level of purity is at least about 95%, more usually at least about 96%, about 97%, about 98%, or higher. For example, the level of purity may be about 98.5%, 99.0%, 99.1%, 99.2%, 99.3%, 99.4%, 99.5%, 99.6%, 99.7%, 99.8%, 99.9%, or higher. Use of such isolated forms avoids the toxicity associated with tobacco, tobacco extracts, alkaloid extracts, and nicotine.

Formulations

Skin care products may be formulated by combining a compound of Formula I and a suitable cosmetic or pharmaceutical vehicle. Non-limiting examples of components conventionally used in skin creams include thickeners; preservatives; lipid-soluble components; methoxycinnamate esters of medium-chain alcohols; benzophenone-3; fragrance; complexes of polyacrylamide, C₁₃-C₁₄ isoparaffin, laureth-7, and water; and colorings. The concentrations of individual components present may vary widely, but often range from about 0.01% to about 10%, more usually from about 0.05% to about 5% (w/w).

Non-limiting examples of thickeners xanthan gum, carrageenan, and combinations thereof. Non-limiting examples of preservatives include methylparaben; butylparaben; propylparaben; a complex of propylene glycol, phenoxyethanol, chlorphenesin, and methylparaben; and combinations thereof. Suitable preservatives are commercially available.

The skin care product may also include lipid-soluble component(s) that provide smoothness. Non-limiting examples of lipid-soluble components include steareth-2; steareth-21; dimethicone; and branched-chain neopentanoate ester selected from the group consisting of octyldodecyl neopentanoate, heptyldodecyl neopentanoate, nonyldodecyl neopentanoate, octylundecyl neopentanoate, heptylundecyl neopentanoate, nonylundecyl neopentanoate, octyltridecyl neopentanoate, heptyltridecyl neopentanoate, and nonyltridecyl neopentanoate. Steareth-2 is polyoxyethylene stearylether with 0.01% butylated hydroxyanisole and 0.005% citric acid added as preservatives. Steareth-21 is polyoxyethylene stearylether with 0.01% butylated hydroxyanisole and 0.005% citric acid added as preservatives.

Other components that may be present include benzophenone-3, which screens out ultraviolet rays. The composition also may include a variety of other components such as coloring agents, fragrance, and the like.

In some cases, the pH of the formulation may be adjusted with acceptable acids, bases or buffers. The compositions also may contain diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylsulfoxide (DMSO) dimethylformamide, oils, glycerol, tetrahydrofurfuryl alcohol, polyethylene glycols and fatty acid esters of sorbitan, and mixtures thereof.

In some embodiments, an isolated form of a compound of I, IA, or IB (e.g., anatabine, S-(−)-anatabine, or R-(+)-anatabine) or salt thereof is provided in a liquid or semisolid form (e.g., liquid, paste, gel, cream, lotion, etc.) for topical application. In some aspects, the skin care product may be applied topically to reduce inflammation, redness, or irritation. For example, the skin care product may be topically administered to reduce the appearance of redness and/or to reduce the appearance of dark circles on the skin. In other aspects, the skin care product may be applied topically to treat an autoimmune and/or dermatological condition such as acne, psoriasis, and/or rosacea.

The following examples illustrate but do not limit the scope of the disclosure set forth above.

### EXAMPLE 1

This example illustrates a skin cream containing anatabine citrate. A skin cream was prepared by combining anatabine citrate with water and thickener, preservative, and lipid-soluble components to yield a cream containing 10.8% anatabine citrate (w/w) and about 56.5% water (w/w).

### EXAMPLE 2

This example illustrates treating acne-rosacea patients with the skin cream of Example 1. For the duration of a 30-day study, the 12 patients washed their faces twice daily with CETAPHIL® facial cleanser and topically applied the skin cream of Example 1 twice daily.

On day 1, patients came to the clinic for baseline assessments, had photographs taken, and received instructions. Clinic staff called patients approximately two weeks later to ask about their experience using the cream. Patients returned to the clinic approximately 30 days later for final assessments and photographs.

Patients were not permitted to use any other skin care products or take any other medications from 48 hours before starting the cream until the end of the one-month study period.

100% of patients saw skin improvement within two weeks. 100% of patients saw improvement in skin redness. 100% of patients said they experienced a smoother skin texture, better hydration, and no irritation. No patients reported any complications.

### EXAMPLE 3

This example illustrates treating rosacea patients with the skin cream of Example 1. For the duration of the 30-day study period, the 10 patients topically applied the Example 1 skin cream twice daily.

On Day 1, patients came to the clinic for baseline assessment, had photographs taken, and received instructions. Clinic staff called patients approximately two weeks later to ask about their experience using the cream. Patients returned to the clinic approximately 30 days later for final assessments and photographs.

Rosacea improved in 7 of the 10 patients, while there was no improvement in two patients, and worsening in one patient.

The study patients also self-assessed their rosacea. Five patients reported rosacea improvement, four patients reported no improvement, and one patient reported worsening of their skin.

While particular embodiments have been described and illustrated, it should be understood that the invention is not limited thereto since modifications may be made by persons skilled in the art. The present application contemplates any and all modifications that fall within the spirit and scope of the underlying invention disclosed and claimed herein.

What is claimed is:

1. A method of treating a skin disorder comprising administering a composition containing a therapeutically effective amount of an isolated form of a compound of Formula I: 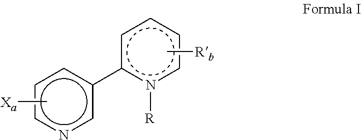
wherein:
R represents hydrogen or C₁-C₅ alkyl;
R′ represents hydrogen or C₁-C₇ alkyl;
X represents halogen or C₁-C₇ alkyl;
“a” represents an integer from 0-4;
“b” represents an integer from 0-8; and
the dotted line within the piperidine ring represents (i) a carbon/carbon or carbon/nitrogen double bond and a carbon/carbon double bond conjugated thereto, or (ii) a carbon/nitrogen double bond;
or a pharmaceutically acceptable salt thereof; and
a pharmaceutically acceptable vehicle therefor.

2. The method of claim 1, wherein the compound of Formula I is anatabine or a salt thereof.

3. The method of claim 1, wherein the compound of Formula I is anatabine citrate.

4. The method of claim 1, wherein the composition is administered topically.

5. The method of claim 1, wherein the skin disorder is selected from the group consisting of psoriasis, rosacea, and acne.

6. The method of claim 5, wherein the skin disorder is psoriasis.

7. The method of claim 5, wherein the skin disorder is rosacea.

8. The method of claim 5, wherein the skin disorder is acne.

9. A method of treating psoriasis comprising administering a composition containing a therapeutically effective amount of anatabine or a pharmaceutically acceptable salt thereof.

10. The method of claim 9, wherein the pharmaceutically acceptable salt is anatabine citrate.

11. The method of claim 10, wherein the composition is administered topically.
